(19) United States
(12) Patent Application Publication
(10) Pub. No.: US 2025/0213785 A1
(43) Pub. Date: Jul. 3, 2025

(54) EXTENDED WEAR INFUSION SET WITH INCREASED INSULIN PRESERVATIVE RETENTION

(71) Applicant: Tandem Diabetes Care, Inc., San Diego, CA (US)

(72) Inventors: Amrith Karunaratne, Irvine, CA (US); Kennie Hsu, Tustin, CA (US); Keith Oberg, San Diego, CA (US)

(21) Appl. No.: 18/398,543

(22) Filed: Dec. 28, 2023

(51) Int. Cl. A61M 5/142

(52) U.S. Cl. CPC A61M 5/14244; A61M 2005/14252; A61M 2205/02; A61M 2209/088

(57) ABSTRACT

Disclosed herein are improved infusion sets that incorporate halogenated siloxane materials within the composition of an infusion set septum. Such configurations maintain the mechanical robustness of using elastomeric materials for a fluidic septum while providing barrier properties that prevent phenolic preservative from being absorbed out of the infusion set. Infusion sets as disclosed herein are particularly suitable for maintaining insulin's anti-microbial effectiveness over extended wear (e.g., 7 days) to maintain performance of the infusion set during extended wear and reduce the risk of insulin aggregate propagation.

### TECHNICAL FIELD

The present disclosure relates infusion sets that facilitate injection of medicament into a body of a patient.

### BACKGROUND

There are a wide variety of medical treatments that include the administration of a therapeutic fluid in precise, known amounts at predetermined intervals. Devices and methods that are directed to the delivery of such fluids, which may be liquids or gases, are known in the art.

One category of such fluid delivery devices includes insulin injecting pumps developed for administering insulin to patients afflicted with type I, or in some cases, type II diabetes. Some insulin injecting pumps are configured as portable or ambulatory infusion devices can provide continuous subcutaneous insulin injection and/or infusion therapy as an alternative to multiple daily injections of insulin via a syringe or an insulin pen. Such pumps are worn by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

Some portable infusion pumps deliver medicament to patients through infusion sets that include tubing extending from the pump and a cannula with an associated needle that penetrates the patient's skin at an infusion site to allow infusion of the medicament through the cannula and into the patient. Such pumps can be worn on the body or carried near the body (e.g., in the user's pocket) with the infusion site situated on the patient's body and connected with the pump via the tubing. Other pumps that are worn directly on the body can deliver medicament through a cannula that extends directly beneath the pump.

If a patient leaves the cannula injected at the injection site at one location for too long a period of time, unwanted side effects such as infection and the accumulation of fat and scar tissue can result. Therefore, patients are often instructed to rotate infusion sites to avoid or minimize side effects. Depending on the type of cannula used, the general physiological response of the patient with regard to insulin absorption, and other factors, the time needed between insulin site rotations can vary. Often, sites are rotated every 24-48 hours or every 48-72 hours. Extended wear infusion sets that can be worn for a longer period of time, such as for example, 7 days, are also being developed.

The insulin delivered by such pumps, including insulin analogs, is an inherently unstable molecule. In addition to chemical changes that can occur as the result of general acid hydrolysis, disulfide scrambling, and other chemical transformations, insulin can be prone to self-associate and precipitate from solution under certain conditions. To counteract this physical instability of insulin, many insulin formulations have been optimized to inhibit insulin precipitation during storage For example, insulin formulations often include phenolic preservatives. Phenolic preservatives are important to maintain within a formulation because they induce an aggregation-resistant conformation (R₆) when they complex with insulin.

Preservative loss in an insulin delivery system is often a two-stage process that includes: (1) absorption of the preservative into fluid-path materials; and (2) evaporation of the fluid preservative from these materials into the air. The absorption rate is generally important in the short term, as well-chosen materials will saturate with preservative rapidly. After the material is saturated, preservative loss from drug product is driven by the rate of preservative diffusion through the material and evaporation into the surrounding environment. Because of this, preservative loss will be driven by residence time of drug product in a component, diffusion rate of preservative through materials, and material thickness.

As noted above, extended wear infusion sets are being developed that may remain in place on the user for longer periods than traditional infusion sets, such as for 7 days. As such, the absorption of the preservatives in insulin into material in the fluid path, already a concern for traditional infusion sets, has become an even greater concern given the longer time period over which insulin will remain within the infusion sets in contact with the infusion set materials. Insulin degradation and precipitation that may result can lead to occlusions in the fluid path and increased tissue inflammation at the infusion site. Design features that maintain insulin stability are critical to providing reliable and safe insulin infusion therapy.

### SUMMARY

Disclosed herein are improved infusion sets that incorporate halogenated siloxane materials within the composition of an infusion set septum. Such configurations maintain the mechanical robustness of using elastomeric materials for a fluidic septum while providing barrier properties that prevent phenolic preservative from being absorbed out of the infusion set. Infusion sets as disclosed herein are particularly suitable for maintaining insulin's anti-microbial effectiveness over extended wear (e.g., 7 days) to maintain performance of the infusion set during extended wear and reduce the risk of insulin aggregate propagation.

In an embodiment, an infusion set configured to facilitate delivery of insulin from an infusion pump to a user can include an infusion hub configured to be placed on a body of the user, infusion tubing configured to transfer insulin form the infusion pump to the infusion hub, a cannula extending from the infusion hub into the body of the user and a septum facilitating transfer of the insulin from the infusion tubing to the cannula. The septum can comprise a halogenated siloxane material.

In an embodiment, an ambulatory infusion pump system can include a reservoir configured to contain insulin, a cannula extending into a body of the user and a septum facilitating transfer of the insulin from the reservoir to the cannula. The septum can comprise a halogenated siloxane material.

In an embodiment, a septum for use in facilitating delivery of insulin to a user can comprise a halogenated siloxane material.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A-1B depict an ambulatory infusion pump with an infusion set according to an embodiment of the disclosure.

FIGS. 2A-2C depict an ambulatory infusion pump according to an embodiment of the disclosure.

FIG. 3 depicts an infusion set according to an embodiment of the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

The following detailed description should be read with reference to the drawings in which similar elements in different drawings are numbered the same. The drawings, which are not necessarily to scale, depict illustrative embodiments and are not intended to limit the scope of the invention.

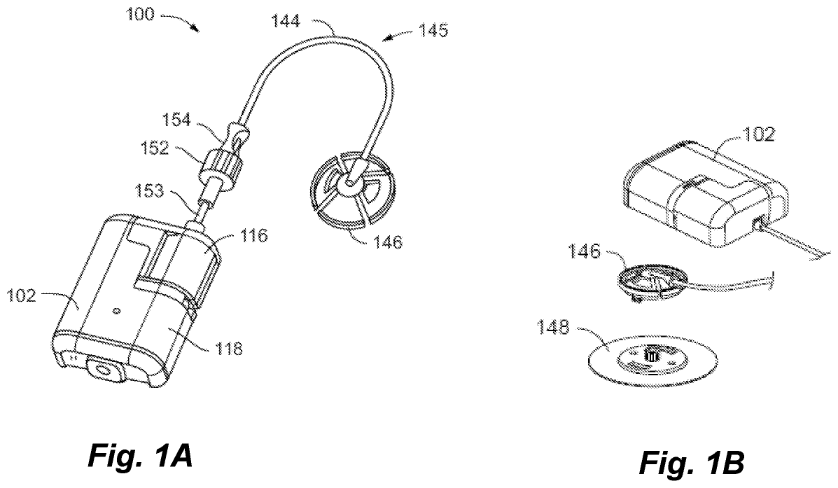

FIGS. 1A-1B depict an exemplary medical device that can be used with embodiments of the disclosure. In this embodiment, the medical device is configured as a pump 102, such as an infusion pump, that can include a pumping or delivery mechanism and a reservoir for delivering a medicament to a patient. In one embodiment, the medical device can be a portable pump configured to deliver insulin to a patient. Further details regarding such pump devices can be found in U.S. Pat. Nos. 8,287,495, 10,279,107 and 10,864,318, each of which is incorporated herein by reference in its entirety. In other embodiments, the medical device can be an infusion pump configured to deliver one or more additional or other medicaments to a patient.

As depicted in FIGS. 1A-1B, pump system 100 can include a pump 102 and an infusion set 145. In embodiments, pump system 100 can include a drive unit 118 and a cartridge 116 having a short length of tubing 153 and a connector 152 extending therefrom. Infusion set 145 can include tubing 144 extending between a connector 154 and a site connector 146. Connector 154 on infusion set 145 can be configured to couple to pump 102 at connector 152 of pump 102. As shown in FIG. 1B, site connector 146 can be configured to be attached to an infusion hub 148 at and infusion site on a user through which medicament from the pump is delivered to a patient through a cannula extending from the infusion hub 148 into the user's skin.

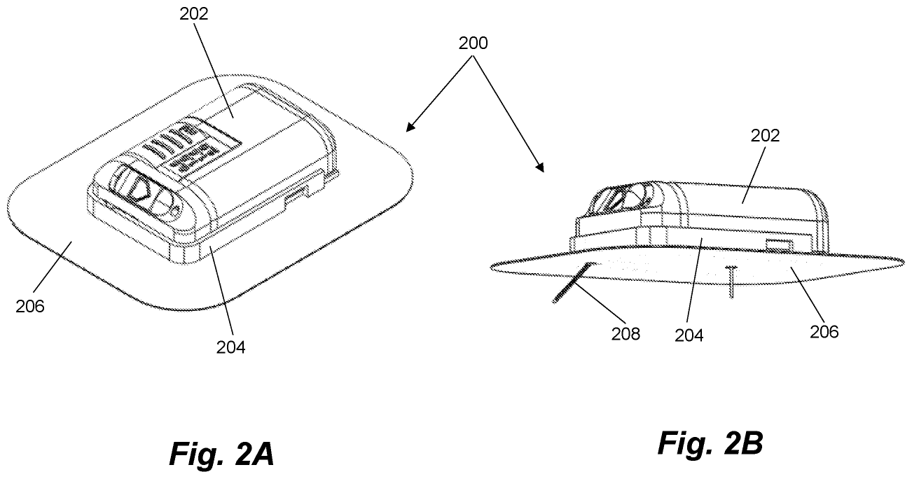

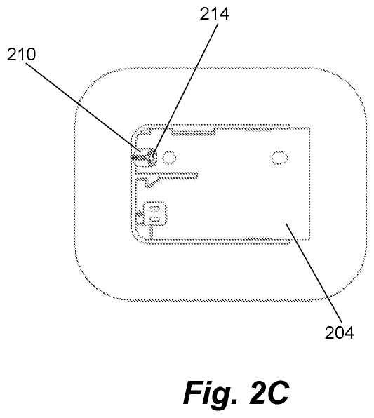

FIGS. 2A-2C depict an infusion pump system 200 according to another embodiment of the disclosure. System 200 can include an infusion pump 202 configured as a patch pump that is worn directly on the body of the user with an adhesive patch 206. Pump 202 can be affixed to the body of the user with an adhesive patch 206 carrying a tray 204 thereon that releasably attaches the pump 202 to the tray 204. Pump 202 can be configured to deliver medicament to the user through a cannula 208 extending directly beneath the pump 202 and adhesive patch 206. Referring to FIG. 2C, in embodiments tray 204 can include a cannula port 210 through which cannula 208 is inserted. The cannula port 210 can contain a septum 214 therein that facilitates transfer of insulin from the pump 202 to the cannula 208.

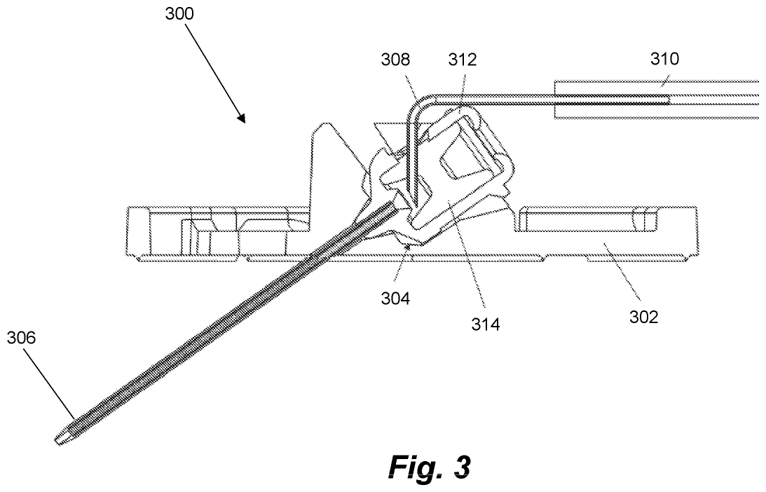

FIG. 3 depicts an infusion hub 300 of an infusion set according to an embodiment of the disclosure. Hub 300 can include a patch 302 or other mechanism configured to adhere to the patient and a barrel 304 connected to a cannula 306. A fluid introducer needle 308 can interface tubing 310 with the hub 300. The barrel 304 can include a mechanical housing 312 configured to house a septum 314. The fluid introducer needle 308 can be configured to pierce the septum 314 to deliver fluid from the tubing 310 to the cannula 306. The fluid path for the insulin medication, therefore, can run from a reservoir of a pump through infusion tubing extending from the pump, through one or more connectors connecting the infusion tubing of the pump with tubing 310 of an infusion set, through the tubing 310 of the infusion set, to the barrel 304 and to the patient via the cannula 306. Although infusion hub 300 is depicted as being employed with a length of tubing 310 that may interface with tubing extending from a pump such as depicted in FIGS. 1A-1B, the components of infusion hub 300 such as the septum 314, cannula 306, etc. can be adapted to be incorporated into a patch pump system such as the system depicted in FIGS. 2A-2B.

In some embodiments, some or all of the fluid path can include a barrier therein to inhibit preservative evaporation and/or loss. The barrier can be, for example, a coating or layer positioned along the fluid path (e.g., a coating along tubing). In other embodiments, the barrier can be an entire component (e.g., due to the material of the tubing). In some embodiments, different components can have different types of barriers. Further details regarding such barriers and infusion sets incorporating such barriers can be found in U.S. Patent Publication No. 2022/0226568, which is hereby incorporated herein by reference in its entirety.

While infusion sets have been designed to minimize preservative evaporation or loss from insulin within the infusion sets, those developments have focused on minimizing such loss in the infusion tubing and/or cannula. However, the septum is the point of insulin transfer from the tubing to the cannula and preservatives can also be lost while insulin is in contact with the septum prior to delivery of the insulin into the cannula. The septum in an infusion set is generally comprised of an elastomeric material such as silicone because such materials accomplish the mechanical sealing requirements of a septum. However, such materials are susceptible to preservative loss such that the septum provides another area where significant leaching of insulin preservatives can occur. Embodiments disclosed herein provide improved septum designs that minimize loss of preservatives in insulin in contact with the septum of an infusion set.

Infusion sets and/or fluid paths for patch pumps according to embodiments disclosed herein can include a septum that is comprised of a halogenated siloxane material. In some embodiments, the septum body can be formed from halogenated siloxane. In other embodiments, a septum formed from silicone or other standard septum materials can be coated with halogenated siloxane such that insulin that contacts the septum will interface with the halogenated siloxane coating rather than the silicone or other material forming the body of the septum. Such configurations maintain the mechanical robustness of using elastomeric material for a fluidic septum, while providing barrier properties that prevent phenolic preservative from being absorbed out of the infusion set.

In embodiments, septum 314 of infusion hub 300 depicted in FIG. 3 can be comprised of a halogenated siloxane. In some embodiments, infusion hub 300 may be the infusion hub 148 of the infusion set as depicted in FIGS. 1A-1B. In other embodiments, a septum such as septum 214 disposed in tray 204 of patch pump system 200 depicted in FIGS. 2A-2B can be comprised of a halogenated siloxane. Septa comprised of halogenated siloxane can further be employed in any type of infusion set, infusion pump, etc. that employs a fluidic, sealable septum.

Preparation of halogenated siloxane materials involves a chemical reaction that includes introduction of one or more halogens into a siloxane material. Halogens are nonmetallic elements that constitute group 17 of the periodic table and include fluorine, chlorine, bromine, iodine, astatine and tennessine. Halogens are the periodic table's most electronegative elements and when incorporated with siloxane functional groups the inventors have found repel phenolic preservative from leaching out of the septum. The inventors have found through testing with a high-performance liquid chromatography machine that septa comprising a halogenated siloxane are at least 5 times less permeable to preservative than standard elastomeric materials such as silicon used for an infusion set septum. This configuration enables maintenance of insulin's anti-microbial effectiveness before being injected into the user's body over extended-wear (e.g., 7 days) insulin delivery through a single infusion set or fluid path. Such a configuration is also particularly useful in pediatric or other applications where a low dose and/or low rate of insulin is delivered such that insulin will remain in contact with the septum for a longer period of time.

In embodiments, an infusion set configured to facilitate delivery of insulin from an infusion pump to a user can include an infusion hub configured to be placed on a body of the user, infusion tubing configured to transfer insulin form the infusion pump to the infusion hub, a cannula extending from the infusion hub into the body of the user and a septum facilitating transfer of the insulin from the infusion tubing to the cannula. The septum can comprise a halogenated siloxane material.

In some embodiments, the septum comprises a septum body.

In some embodiments, the septum body comprises the halogenated siloxane material.

In some embodiments, the septum body is coated with the halogenated siloxane material.

In some embodiments, a transfer needle extends from the infusion tubing to pierce the septum.

In embodiments, an ambulatory infusion pump system can include a reservoir configured to contain insulin, a cannula extending into a body of the user and a septum facilitating transfer of the insulin from the reservoir to the cannula. The septum can comprise a halogenated siloxane material.

In some embodiments, the septum comprises a septum body.

In some embodiments, the septum body comprises the halogenated siloxane material.

In some embodiments, the septum body is coated with the halogenated siloxane material.

In some embodiments, an infusion hub can be configured to be placed on a body of the user and infusion tubing can be configured to transfer the insulin from the reservoir to the septum.

The cannula can extend from the infusion hub into the body of the user.

In some embodiments, a transfer needle can extend from the infusion tubing to pierce the septum.

In some embodiments, an infusion pump contains the reservoir and a pump holder is configured to be worn on the body of the user to releasably hold the infusion pump.

In some embodiments, the pump holder includes a cannula port from which the cannula extends into the body of the user.

In some embodiments, the septum is disposed in the cannula port.

In embodiments, a septum for use in facilitating delivery of insulin to a user can comprise a halogenated siloxane material.

In some embodiments, the septum comprises a septum body.

In some embodiments, the septum body comprises the halogenated siloxane material.

In some embodiments, the septum body is coated with the halogenated siloxane material.

Although the infusion pump embodiments herein are specifically described primarily with respect to the delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; 11,464,908; and 11,654,236 and commonly owned U.S.

Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0368430; 2020/0372995; 2021/0001044; 2021/0113766; 2021/0353857; 2022/0062553; 2022/0139522; 2022/0223250; 2022/0233772; 2022/0233773; 2022/0238201; 2022/0265927; 2023/0034408; 2022/0344017; 2022/0370708; 2022/0037465; 2023/0040677; 2023/0047034; 2023/0113545 and 2023/0113755 and commonly owned U.S. patent application Ser. Nos. 17/368,968; 17/896,492; 18/011,060; 18/071,814; 18/071,835; 18/075,029; 18/090,788 18/115,316; and Ser. No. 18/139,391.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing detailed description.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1. An infusion set configured to facilitate delivery of insulin from an infusion pump to a user, comprising:
an infusion hub configured to be placed on a body of the user;
infusion tubing configured to transfer insulin form the infusion pump to the infusion hub;
a cannula extending from the infusion hub into the body of the user; and
a septum facilitating transfer of the insulin from the infusion tubing to the cannula, wherein the septum comprises a halogenated siloxane material.

2. The infusion set of claim 1, wherein the septum comprises a septum body.

3. The infusion set of claim 2, wherein the septum body comprises the halogenated siloxane material.

4. The infusion set of claim 2, wherein the septum body is coated with the halogenated siloxane material.

5. The infusion set of claim 1, further comprising a transfer needle extending from the infusion tubing configured to pierce the septum.

6. An ambulatory infusion pump system, comprising:
a reservoir configured to contain insulin;
a cannula extending into a body of the user; and
a septum facilitating transfer of the insulin from the reservoir to the cannula, wherein the septum comprises a halogenated siloxane material.

7. The ambulatory infusion pump system of claim 6, wherein the septum comprises a septum body.

8. The ambulatory infusion pump system of claim 7, wherein the septum body comprises the halogenated siloxane material.

9. The ambulatory infusion pump system of claim 7, wherein the septum body is coated with the halogenated siloxane material.

10. The ambulatory infusion pump system of claim 6, further comprising an infusion hub configured to be placed on a body of the user and infusion tubing, wherein the infusion tubing is configured to transfer the insulin from the reservoir to the septum and the cannula extends from the infusion hub into the body of the user.

11. The ambulatory infusion pump system of claim 10, further comprising a transfer needle extending from the infusion tubing configured to pierce the septum.

12. The ambulatory infusion pump system of claim 6, further comprising an infusion pump containing the reservoir and a pump holder configured to be worn on the body of the user to releasably hold the infusion pump.

13. The ambulatory infusion pump system of claim 12, wherein the pump holder includes a cannula port from which the cannula extends into the body of the user.

14. The ambulatory infusion pump system of claim 13, wherein the septum is disposed in the cannula port.

15. A septum for use in facilitating delivery of insulin to a user, the septum comprising a halogenated siloxane material.

16. The septum of claim 15, comprising a septum body.

17. The septum of claim 16, wherein the septum body comprises the halogenated siloxane material.

18. The septum of claim 16, wherein the septum body is coated with the halogenated siloxane material.
